(19) United States
(12) Patent Application Publication
(10) Pub. No.: US 2022/0220500 A1
(43) Pub. Date: Jul. 14, 2022

(54) METHOD FOR THE PRODUCTION OF AN ANTIBODY

(71) Applicant: Hoffmann-La Roche Inc., Little Falls, NJ (US)

(72) Inventors: Ulrich Goepfert, Penzberg (DE); Stefan Klostermann, Neuried (DE); Katharina Lutz, Basel (CH); Stefan Seeber, Sindelsdorf (DE)

(73) Assignee: Hoffmann-La Roche Inc., Little Falls, NJ (US)

(21) Appl. No.: 17/559,934

(22) Filed: Dec. 22, 2021

Related U.S. Application Data
(63) Continuation of application No. PCT/EP2020/067770, filed on Jun. 25, 2020.

(30) Foreign Application Priority Data
Jun. 28, 2019 (EP) 19183171.8

(51) Int. Cl. C12N 15/85; C07K 16/00

(52) U.S. Cl. CPC C12N 15/85; C07K 16/00; C07K 2317/14; C12N 2820/60; C07K 2317/24; C07K 2317/52; C07K 2317/21

(57) ABSTRACT

Herein is reported a method for producing an IgG1 antibody by cultivating a CHO cell comprising/transfected with one or more (exogenous) nucleic acids encoding the antibody (and expressing the antibody), wherein in the nucleic acid encoding the heavy chain variable domain non-paired splice sites are removed and in the nucleic acid encoding the heavy chain constant region these are not removed.

### CROSS-REFERENCE TO RELATED APPLICATIONS

This application is a continuation of International Application No. PCT/EP2020/067770 having an International filing date of Jun. 25, 2020, which claims benefit of priority to European Patent Application No. 19183171.8, filed Jun. 28, 2019, all of which are incorporated by reference in their entirety.

### SEQUENCE LISTING

This application contains a Sequence Listing which has been submitted electronically in ASCII format and is hereby incorporated by reference in its entirety. Said ASCII copy, created on Dec. 20, 2021, is named P35209-US_Sequence_Listing.txt and is 1,403 bytes in size.

### FIELD OF INVENTION

Herein is reported a method for the production of an antibody wherein the encoding nucleic acid is optimized with respect to donor splice sites only in the part encoding the variable domain.

### BACKGROUND OF THE INVENTION

Cannarozzi, G., et al. report the role of codon order in translation dynamics (Cell 141 (2010) 355-367). The cause and consequence of codon bias is reported by Plotkin, J. B. and Kudla, G. (Nat. Rev. Gen. 12 (2011) 32-42). Weygand-Durasevic, I. and Ibba, M., report new roles for codon usage (Science 329 (2010) 1473-1474). Overlapping codes within protein-coding sequences is reported by Itzkovitz, S., et al. (Gen. Res. 20 (2010) 1582-1589).

In WO 97/11086 high level expression of proteins is reported. Plant polypeptide production is reported in WO 03/70957. In WO 03/85114 a method for designing synthetic nucleic acid sequences for optimal protein expression in a host cell. Codon pair optimization is reported in U.S. Pat. No. 5,082,767. In WO 2008/000632 a method for achieving improved polypeptide expression is reported. A codon optimization method is reported in WO 2007/142954 and U.S. Pat. No. 8,128,938.

Watkins, N. E., et al., report nearest-neighbor thermodynamics of deoxyinosine pairs in DNA duplexes (Nucl. Acids Res. 33 (2005) 6258-6267).

In WO 2013/156443 a method for the expression of polypeptides using modified nucleic acids is reported.

Zhang, M. Q. reported statistical features of human exons and their flanking regions (Hum. Mol. Genet. 7 (1998) 919-932).

The splicing of mRNA is regulated by the occurrence of a donor splice site in combination with an acceptor splice site, which are located at the 5′ end and 3′ end of an intron, respectively. According to Watson et al. (Watson et al. (Eds), Recombinant DNA: A Short course, Scientific American Books, distributed by W.H. Freeman and Company, New York, N.Y., USA (1983)) are the consensus sequence of the 5′ donor splice site ag|gtragt (exon|intron) and of the 3′ acceptor splice site (y)ₙNcag|g (intron|exon) (r=purine base; y=pyrimidine base; n=integer; N=any natural base).

In 1980 first articles dealing with the origin of secreted and membrane bound forms of immunoglobulins have been published. The formation of the secreted (sIg) and the membrane bound (mIg) isoform results from alternative splicing of the heavy chain pre-mRNA. In the mIg isoform a donor splice site in the exon encoding the C-terminal domain of the secreted form (i.e. the C_{H}3 or C_{H}4 domain, respectively) and an acceptor splice site located at a distance downstream thereof are used to link the constant region with the downstream exons encoding the transmembrane domain.

A method to prepare synthetic nucleic acid molecules having reduced inappropriate or unintended transcriptional characteristics when expressed in a particular host cell is reported in WO 2002/016944. In WO 2006/042158 are reported nucleic acid molecules modified to enhance recombinant protein expression and/or reduce or eliminate mis-spliced and/or intron read through by products. Magistrelli, G., et al. reported optimizing assembly and production of native bispecific antibodies by codon de-optimization (MABS 9 (2016) 231-239).

WO 2015/128509 reported expression constructs and methods for selecting host cells expressing polypeptides.

WO 2009/003623 reported a heavy chain mutant leading to improved immunoglobulin production.

### SUMMARY OF THE INVENTION

For the production of a therapeutic or diagnostic antibody high expression yields are the aim. One general option to achieve good expression rates is to optimize the codon usage of the encoding nucleic acids at first by adjusting it to the codon usage of the cell intended to express the exogenous nucleic acid. Such a codon adaptation or optimization can be done based on different established protocols.

But during such a codon adaptation and optimization process, e.g. non-paired splices sites, especially non-paired donor splice sites, can be generated unintentionally de novo. That is, e.g., during codon optimization a new donor splice site sequence is generated in the codon usage optimized nucleic acid by inadvertently generating a sequence motif inside the codon-optimized nucleic acid that follows a donor splice site consensus sequence. Such an event can take place independently on the organization of the codon usage optimize nucleic acid, i.e. it is possible for both cDNA as well as genomically organized nucleic acids. It is in fact an unintended side-result of the codon usage optimization process. Such a new donor splice site is an additional artificial donor splice site and it does not have an associated target acceptor splice site. Thus, such non-paired donor splice sites can give rise to splicing events with a random, i.e. non-defined, acceptor splice site present somewhere in the transcribed mRNA. Thereby the expression yield is reduced due to the formation of by-products.

The invention is based, at least in part, on the unexpected finding that the removal of non-paired donor splice sites in a codon usage optimized antibody heavy chain encoding nucleic acid needs only to be performed in the part of the nucleic acid encoding the variable domain of the heavy chain but not in the part encoding the constant region, i.e. for the constant region, e.g., the germline or wild-type human nucleic acid sequence can be used. Thereby the expression yield of the antibody heavy chain with correct length can be increased or becomes possible at all.

The current invention is based, at least in part, on the finding that the introduction of amino acid sequence silent nucleotide changes (mutations) in the non-paired donor splice site consensus sequence NGGTA(G)AG (SEQ ID NO: 01) only in the codon optimized nucleic acid encoding the variable domain of an antibody heavy chain is sufficient to improve the expression yield.

One aspect of the current invention is a method for producing an antibody by cultivating a mammalian cell comprising/transfected with one or more (exogenous) nucleic acids encoding the antibody heavy chain and the antibody light chain (and expressing the antibody),
wherein the one or more (exogenous) nucleic acids are codon usage optimized for the codon usage of human cells and/or for the codon usage of the mammalian cell,
wherein in the nucleic acid encoding the heavy chain variable domain at least one (artificial) non-paired donor splice site is removed and, optionally, in the (human wild-type or human or hamster codon usage optimized) nucleic acid sequence encoding the heavy chain constant region (artificial) non-paired donor splice sites are not removed.

In one embodiment the antibody is an antibody of the human IgG1 subclass. In one embodiment the antibody is a humanized antibody of the human IgG1 subclass. In one embodiment the constant region of the antibody comprises mutations suitable to induce heterodimerization or modify Fc-receptor binding

In one embodiment the mammalian cell is a CHO cell.

In one embodiment the transfection is a transient transfection.

In one embodiment the one or more (exogenous) nucleic acids encoding the antibody are all cDNA.

In one embodiment the one or more (exogenous) nucleic acids encoding the antibody heavy chain and/or the antibody light chain are genomically organized DNA, i.e. have an intron-exon-organization.

In one embodiment the removal of the non-paired donor splice sites is by introducing an amino acid silent change (mutation) in the amino acid sequence NGGTA(G)AG (SEQ ID NO: 01). In one embodiment the amino acid sequence silent nucleotide change is introduced in the codon NGG or the codon GGT or the codon GTA(G) of SEQ ID NO: 01.

In one embodiment the codon usage optimization is done based on the human codon usage or based on the chinese hamster codon usage.

In one embodiment the nucleic acid encoding the antibody light chain is codon usage optimized, i.e. the variable domain and the constant region is codon usage optimized.

In one embodiment in the complete light chain encoding nucleic acid non-paired donor splice sites are removed

In one embodiment the transfection is a stable transfection.

In one embodiment the method comprises the following steps:
a) cultivating the mammalian cell, and
b) recovering the antibody from the cell or the cultivation medium.

One aspect of the current invention is the use of the removal of non-paired donor splice sites only in a part of a human or hamster codon-usage optimized nucleic acid sequence encoding an antibody for reducing mis-splicing or/and increasing antibody expression yield when said nucleic acid is used to produce the antibody in CHO cells, whereby the part is the part that encodes the heavy chain variable domain.

In one embodiment non-paired donor splice sites are not removed in the part of the nucleic acid encoding the heavy chain constant region.

In one embodiment further in the part of the nucleic acid encoding the light chain non-paired donor splice sites are removed.

In one embodiment the removal of the non-paired splice sites is by introducing an amino acid silent mutation in the nucleotide sequence NGGTA(G)AG (SEQ ID NO: 01).

In one embodiment the removal of the non-paired splice sites is by introducing an amino acid silent mutation in the nucleotide sequence NGGTA(G)AG (SEQ ID NO: 01) in the codon NGG or the codon GGT or the codon GTA(G).

In one embodiment of all aspects and embodiments the non-paired (donor) splice site is an artificial non-paired (donor) splice site.

In one embodiment of all aspects and embodiments the non-paired (donor) splice site is an artificial non-paired (donor) splice site and has been generated during codon optimization.

### DETAILED DESCRIPTION OF EMBODIMENTS OF THE INVENTION

For the production of a therapeutic or diagnostic antibody high expression yields are the aim. One option to achieve good expression rates is to optimize the codon usage of the encoding nucleic acids at first and adjust it to the codon usage of the cell intended to express the exogenous nucleic acid. This codon adaptation or optimization can be done based on different established protocols.

But during such a codon adaptation and optimization process, e.g. non-paired splices sites can be generated de novo. That is, during codon optimization a new donor splice site sequence is generated in the codon usage optimized nucleic acid. This is independent on the organization of the codon usage optimize nucleic acid, i.e. it is possible for both cDNA or genomically organized nucleic acids. It is in fact an unintended side-result of the codon usage optimization process. As such a new donor splice site is an additional artificial donor splice site it does not have an associated target acceptor splice site. Thus, such non-paired donor splice sites can give rise to splicing events with a random, i.e. non-defined, acceptor splice site present somewhere in the transcribed mRNA. Thereby the expression yield is reduced.

The invention is based, at least in part, on the unexpected finding that the removal of non-paired donor splice sites in a codon usage optimized antibody heavy chain encoding nucleic acid needs only to be performed in the part of the nucleic acid encoding the variable domain of the heavy chain but not in the part encoding the constant region, i.e. for the constant region, e.g., the germline or wild-type human nucleic acid sequence can be used.

The current invention is based, at least in part, on the finding that the introduction of amino acid sequence silent nucleotide changes (mutations) in the non-paired donor splice site consensus sequence NGGTA(G)AG (SEQ ID NO: 01) only in the codon optimized nucleic acid encoding the variable domain of an antibody heavy chain is sufficient to improve the expression yield.

### Definitions

Methods and techniques useful for carrying out the current invention are known to a person skilled in the art and are described e.g. in Ausubel, F. M., ed., Current Protocols in Molecular Biology, Volumes I to III (1997), and Sambrook et al., Molecular Cloning: A Laboratory Manual, Second Edition, Cold Spring Harbor Laboratory Press, Cold Spring Harbor, N.Y. (1989). As known to a person skilled in the art enables the use of recombinant DNA technology the production of numerous derivatives of a nucleic acid and/or polypeptide.

Such derivatives can, for example, be modified in one individual or several positions by substitution, alteration, exchange, deletion, or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA). The use of recombinant technology enables a person skilled in the art to transform various host cells with heterologous nucleic acid(s). Although the transcription and translation, i.e. expression, machinery of different cells use the same elements, cells belonging to different species may have among other things a different so-called codon usage. Thereby identical polypeptides (with respect to amino acid sequence) may be encoded by different nucleic acid(s). Also, due to the degeneracy of the genetic code, different nucleic acids may encode the same polypeptide.

The term “about” denotes that the thereafter following value is no exact value but is the center point of a range that is +/−10% of the value, or +/−5% of the value, or +/−2% of the value, or +/−1% of the value. If the value is a relative value given in percentages the term “about” also denotes that the thereafter following value is no exact value but is the center point of a range that is +/−10% of the value, or +/−5% of the value, or +/−2% of the value, or +/−1% of the value, whereby the upper limit of the range cannot exceed a value of 100%.

The term “amino acid” as used within this application denotes the group of carboxy α-amino acids, which directly or in form of a precursor can be encoded by a nucleic acid. The individual amino acids are encoded by nucleic acids consisting of three nucleotides, so called codons or base-triplets. Each amino acid is encoded by at least one codon. The encoding of the same amino acid by different codons is known as “degeneration of the genetic code”. The term “amino acid” as used within this application denotes the naturally occurring carboxy α-amino acids and is comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “immunoglobulin” herein is used in the broadest sense and encompasses various immunoglobulin structures, including but not limited to monoclonal antibodies, polyclonal antibodies, as well as multispecific antibodies (e.g., bispecific antibodies) or fragments thereof comprising at least a part of a constant domain or region.

As used herein, the term “immunoglobulin” denotes a protein consisting of one or more polypeptides substantially encoded by immunoglobulin genes. This definition includes variants such as mutated forms, i.e. forms with substitutions, deletions, and insertions of one or more amino acids, N-terminally truncated forms, fused forms, chimeric forms, as well as humanized forms. The recognized immunoglobulin genes include the different constant region genes as well as the myriad immunoglobulin variable region genes from, e.g., primates, including humans, and rodents. Monoclonal immunoglobulins are preferred. Each of the heavy and light polypeptide chains of an immunoglobulin may comprise a constant region (generally the carboxyl terminal portion).

The term “monoclonal immunoglobulin” as used herein refers to an immunoglobulin obtained from a population of substantially homogeneous immunoglobulins, i.e. the individual immunoglobulins comprising the population are identical except for possible naturally occurring mutations that may be present in minor amounts. Monoclonal immunoglobulins are highly specific, being directed against a single antigenic site. Furthermore, in contrast to polyclonal immunoglobulin preparations, which include different immunoglobulins directed against different antigenic sites (determinants or epitopes), each monoclonal immunoglobulin is directed against a single antigenic site on the antigen. In addition to their specificity, the monoclonal immunoglobulins are advantageous in that they may be synthesized uncontaminated by other immunoglobulins. The modifier “monoclonal” indicates the character of the immunoglobulin as being obtained from a substantially homogeneous population of immunoglobulins and is not to be construed as requiring production of the immunoglobulin by any particular method.

The term “codon” denotes an oligonucleotide consisting of three nucleotides that is encoding a defined amino acid. Due to the degeneracy of the genetic code most amino acids are encoded by more than one codon. These different codons encoding the same amino acid have different relative usage frequencies in individual host cells. Thus, a specific amino acid is encoded either by exactly one codon or by a group of different codons. Likewise, the amino acid sequence of a polypeptide can be encoded by different nucleic acids. Therefore, a specific amino acid (residue) in a polypeptide can be encoded by a group of different codons, whereby each of these codons has a usage frequency within a given host cell.

As a large number of gene sequences is available for a number of frequently used host cells the relative frequencies of codon usage can be calculated. Calculated codon usage tables are available from e.g. the “Codon Usage Database” (www.kazusa.or.jp/codon/), Nakamura, Y., et al., Nucl. Acids Res. 28 (2000) 292.

The codon usage tables for Homo sapiens and hamster have been reproduced from “EMBOSS: The European Molecular Biology Open Software Suite” (Rice, P., et al., Trends Gen. 16 (2000) 276-277, Release 6.0.1, 15.07.2009) and are shown in the following tables. The different codon usage frequencies for the 20 naturally occurring amino acids for E. coli, yeast, human cells, and CHO cells have been calculated for each amino acid, rather than for all 64 codons.

TABLE
Homo sapiens overall codon usage frequency
(encoded amino acid | codon | usage frequency [%])

| | | |
|---|---|---|
| Ala | GCG | 10 |
| Ala | GCA | 22 |
| Ala | GCT | 27 |
| Ala | GCC | 41 |
| Arg | AGG | 20 |
| Arg | AGA | 20 |
| Arg | CGG | 20 |
| Arg | CGA | 11 |
| Arg | CGT | 9 |
| Arg | CGC | 20 |
| Asn | AAT | 45 |
| Asn | AAC | 55 |
| Asp | GAT | 46 |
| Asp | GAC | 54 |
| Cys | TGT | 44 |
| Cys | TGC | 56 |
| Gln | CAG | 74 |
| Gln | CAA | 26 |
| Glu | GAG | 58 |
| Glu | GAA | 42 |
| Gly | GGG | 24 |
| Gly | GGA | 25 |
| Gly | GGT | 17 |
| Gly | GGC | 34 |
| His | CAT | 40 |
| His | CAC | 60 |
| Ile | ATA | 15 |
| Ile | ATT | 35 |
| Ile | ATC | 50 |
| Leu | CTG | 42 |
| Leu | CTA | 7 |
| Leu | CTT | 13 |
| Leu | CTC | 20 |
| Leu | TTG | 12 |
| Leu | TTA | 7 |
| Lys | AAG | 59 |
| Lys | AAA | 41 |
| Met | ATG | 100 |
| Phe | TTT | 45 |
| Phe | TTC | 55 |
| Pro | CCG | 11 |
| Pro | CCA | 28 |
| Pro | CCT | 28 |
| Pro | CCC | 33 |
| Ser | AGT | 15 |
| Ser | AGC | 25 |
| Ser | TCG | 6 |
| Ser | TCA | 14 |
| Ser | TCT | 18 |
| Ser | TCC | 23 |
| Thr | ACG | 12 |
| Thr | ACA | 27 |
| Thr | ACT | 24 |
| Thr | ACC | 37 |
| Trp | TGG | 100 |
| Tyr | TAT | 43 |
| Tyr | TAC | 57 |
| Val | GTG | 47 |
| Val | GTA | 11 |
| Val | GTT | 17 |
| Val | GTC | 25 |

TABLE
Hamster overall codon usage frequence
(encoded amino acid | codon | usage frequency [%])

| | | |
|---|---|---|
| Ala | GCG | 9 |
| Ala | GCA | 23 |
| Ala | GCT | 30 |
| Ala | GCC | 38 |
| Arg | AGG | 22 |
| Arg | AGA | 20 |
| Arg | CGG | 19 |
| Arg | CGA | 9 |
| Arg | CGT | 10 |
| Arg | CGC | 19 |
| Asn | AAT | 39 |
| Asn | AAC | 61 |
| Asp | GAT | 39 |
| Asp | GAC | 61 |
| Cys | TGT | 42 |
| Cys | TGC | 58 |
| Gln | CAG | 78 |
| Gln | CAA | 22 |
| Glu | GAG | 64 |
| Glu | GAA | 36 |
| Gly | GGG | 24 |
| Gly | GGA | 25 |
| Gly | GGT | 19 |
| Gly | GGC | 33 |
| His | CAT | 42 |
| His | CAC | 58 |
| Ile | ATA | 15 |
| Ile | ATT | 35 |
| Ile | ATC | 51 |
| Leu | CTG | 44 |
| Leu | CTA | 6 |
| Leu | CTT | 13 |
| Leu | CTC | 19 |
| Leu | TTG | 12 |
| Leu | TTA | 6 |
| Lys | AAG | 67 |
| Lys | AAA | 33 |
| Met | ATG | 100 |
| Phe | TTT | 44 |
| Phe | TTC | 56 |
| Pro | CCG | 7 |
| Pro | CCA | 29 |
| Pro | CCT | 29 |
| Pro | CCC | 34 |
| Ser | AGT | 14 |
| Ser | AGC | 24 |
| Ser | TCG | 5 |
| Ser | TCA | 15 |
| Ser | TCT | 18 |
| Ser | TCC | 24 |
| Thr | ACG | 10 |
| Thr | ACA | 29 |
| Thr | ACT | 21 |
| Thr | ACC | 40 |
| Trp | TGG | 100 |
| Tyr | TAT | 39 |
| Tyr | TAC | 61 |
| Val | GTG | 48 |
| Val | GTA | 11 |
| Val | GTT | 16 |
| Val | GTC | 25 |

The term “expression” as used herein refers to transcription and/or translation processes occurring within a cell. The level of transcription of a nucleic acid sequence of interest in a cell can be determined on the basis of the amount of corresponding mRNA that is present in the cell. For example, mRNA transcribed from a sequence of interest can be quantitated by RT-PCR (qRT-PCR) or by Northern hybridization (see Sambrook, J., et al., 1989, supra). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook, J., et al., 1989, supra).

An “expression cassette” refers to a construct that contains the necessary regulatory elements, such as promoter and polyadenylation site, for expression of at least the contained nucleic acid in a cell.

Expression of a gene is performed either as transient or as permanent expression. The polypeptide(s) of interest are in general secreted polypeptides and therefore contain an N-terminal extension (also known as the signal sequence) which is necessary for the transport/secretion of the polypeptide through the cell membrane into the extracellular medium. In general, the signal sequence can be derived from any gene encoding a secreted polypeptide. If a heterologous signal sequence is used, it preferably is one that is recognized and processed (i.e. cleaved by a signal peptidase) by the host cell. For secretion in yeast for example the native signal sequence of a heterologous gene to be expressed may be substituted by a homologous yeast signal sequence derived from a secreted gene, such as the yeast invertase signal sequence, alpha-factor leader (including Saccharomyces, Kluyveromyces, Pichia, and Hansenula α-factor leaders, the second described in U.S. Pat. No. 5,010,182), acid phosphatase signal sequence, or the C. albicans glucoamylase signal sequence (EP 0 362 179). In mammalian cell expression the native signal sequence of the protein of interest is satisfactory, although other mammalian signal sequences may be suitable, such as signal sequences from secreted polypeptides of the same or related species, e.g. for immunoglobulins from human or murine origin, as well as viral secretory signal sequences, for example, the herpes simplex glycoprotein D signal sequence. The DNA fragment encoding for such a pre-segment is ligated in frame, i.e. operably linked, to the DNA fragment encoding a polypeptide of interest.

The term “cell” or “host cell” refers to a cell into which a nucleic acid, e.g. encoding a heterologous polypeptide, can be or is transfected. The term “cell” includes both prokaryotic cells, which are used for expression of a nucleic acid and production of the encoded polypeptide including propagation of plasmids, and eukaryotic cells, which are used for the expression of a nucleic acid and production of the encoded polypeptide. In one embodiment, the eukaryotic cells are mammalian cells. In one embodiment the mammalian cell is a CHO cell, optionally a CHO K1 cell (ATCC CCL-61 or DSM ACC 110), or a CHO DG44 cell (also known as CHO-DHFR[-], DSM ACC 126), or a CHO XL99 cell, a CHO-T cell (see e.g. Morgan, D., et al., Biochemistry 26 (1987) 2959-2963), or a CHO-S cell, or a Super-CHO cell (Pak, S.C.O., et al. Cytotechnology 22 (1996) 139-146). If these cells are not adapted to growth in serum-free medium or in suspension an adaptation prior to the use in the current method is to be performed. As used herein, the expression “cell” includes the subject cell and its progeny. Thus, the words “transformant” and “transformed cell” include the primary subject cell and cultures derived there from without regard for the number of transfers or subcultivations. It is also understood that all progeny may not be precisely identical in DNA content, due to deliberate or inadvertent mutations. Variant progeny that have the same function or biological activity as screened for in the originally transformed cell are included.

The term “codon-optimized nucleic acid” denotes a nucleic acid encoding a polypeptide that has been adapted for improved expression in a cell, e.g. a mammalian cell, by replacing one, at least one, or more than one codon in a parent polypeptide encoding nucleic acid with a codon encoding the same amino acid residue, e.g. with a different relative frequency of usage in the cell.

The term “non-paired donor splice site” as used herein denotes a donor splice site that on the one hand has been artificially generated inside a nucleic acid sequence, e.g. by codon optimization of the nucleic acid sequence, and that on the other hand has due to its artificial introduction into the nucleic acid sequence no linked acceptor splice site downstream in the nucleic acid sequence. Although being in line with the donor splice site consensus sequence, it does not follow the (biological) splicing principle, i.e. the excision of not-wanted parts of the nucleic acid during processing.

A “gene” denotes a nucleic acid which is a segment e.g. on a chromosome or on a plasmid which can affect the expression of a peptide, polypeptide, or protein. Beside the coding region, i.e. the structural gene, a gene comprises other functional elements e.g. a signal sequence, promoter(s), introns, and/or terminators.

The term “group of codons” and semantic equivalents thereof denote a defined number of different codons encoding one (i.e. the same) amino acid residue. The individual codons of one group differ in their overall usage frequency in the genome of a cell. Each codon in a group of codons has a specific usage frequency within the group that depends on the number of codons in the group. This specific usage frequency within the group can be different from the overall usage frequency in the genome of a cell but is depending (related thereto) on the overall usage frequency. A group of codons may comprise only one codon but can comprise also up to six codons.

The term “overall usage frequency in the genome of a cell” denotes the frequency of occurrence of a specific codon in the entire genome of a cell.

The term “specific usage frequency” of a codon in a group of codons denotes the frequency with which a single (i.e. a specific) codon of a group of codons in relation to all codons of one group can be found in a nucleic acid encoding a polypeptide obtained with a method as reported herein. The value of the specific usage frequency depends on the overall usage frequency of the specific codon in the genome of a cell and the number of codons in the group. Thus, as a group of codons does not necessarily comprise all possible codons encoding one specific amino acid residue the specific usage frequency of a codon in a group of codons is at least the same as its overall usage frequency in the genome of a cell and at most 100%, i.e. it is at least the same but can be more than the overall usage frequency in the genome of a cell if certain codons with low usage frequence are excluded from the group. The sum of specific codon usage frequencies of all members of a group of codons is always about 100%.

The term “amino acid codon motif” denotes a sequence of codons, which all are members of the same group of codons and, thus, encode the same amino acid residue. The number of different codons in an amino acid codon motif is the same as the number of different codons in a group of codons but each codon can be present more than once in the amino acid codon motif. Further, each codon is present in the amino acid codon motif at its specific usage frequency. Therefore, the amino acid codon motif represents a sequence of different codons encoding the same amino acid residue wherein each of the different codons is present at its specific usage frequency, wherein the sequence starts with the codon having the highest specific usage frequence, and wherein the codons are arranged in a defined sequence. For example, the group of codons encoding the amino acid residue alanine comprises the four codons GCG, GCT, GCA and GCC with a specific usage frequency of 32%, 28%, 24% and 16%, respectively (corresponding to a 4:3:3:2 ratio). The amino acid codon motif for the amino acid residue alanine is defined in comprising the four codons GCG, GCT, GCA, and GCC at a ratio of 4:3:3:2, wherein the first codon is GCG. One exemplary amino acid codon motif for alanine is gcg gct gca gcc gcg gct gca gcc gcg gct gca gcg (SEQ ID NO: 06). This motif consists of twelve sequential codons (4+3+3+2=12). Upon the first occurrence of the amino acid residue alanine in the amino acid sequence of a polypeptide the first codon of the amino acid codon motif is used in the corresponding encoding nucleic acid. Upon the second occurrence of alanine the second codon of the amino acid codon motif is used and so on. Upon the thirteenth occurrence of alanine in the amino acid sequence of the polypeptide the codon at the thirteenth, i.e. the last, position of the amino acid codon motif is used in the corresponding encoding nucleic acid. Upon the thirteenth occurrence of the amino acid alanine in the amino acid sequence of the polypeptide again the first codon of the amino acid codon motif is used and so on.

A “nucleic acid” or a “nucleic acid sequence”, which terms are used interchangeably within this application, refers to a polymeric molecule consisting of individual nucleotides (also called bases) a, c, g, and t (or u in RNA), for example to DNA, RNA, or modifications and mixtures thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed (e.g. by mutagenesis), deleted, or added. A nucleic acid can either be isolated, or integrated in another nucleic acid, e.g. in an expression cassette, a plasmid, or the chromosome of a host cell. A nucleic acid is characterized by its nucleic acid sequence consisting of individual nucleotides.

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

A “structural gene” denotes the region of a gene without a signal sequence, i.e. the coding region.

A “transfection vector” is a nucleic acid (also denoted as nucleic acid molecule) providing all required elements for the expression of the in the transfection vector comprised coding nucleic acids/structural gene(s) in a host cell. A transfection vector comprises a prokaryotic plasmid propagation unit, e.g. for E. coli, in turn comprising a prokaryotic origin of replication, and a nucleic acid conferring resistance to a prokaryotic selection agent, further comprises the transfection vector one or more nucleic acid(s) conferring resistance to a eukaryotic selection agent, and one or more nucleic acid encoding a polypeptide of interest. Preferably are the nucleic acids conferring resistance to a selection agent and the nucleic acid(s) encoding a polypeptide of interest placed each within an expression cassette, whereby each expression cassette comprises a promoter, a coding nucleic acid, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “full length antibody” denotes an antibody having a structure substantially similar to that of a native antibody. A full length antibody comprises two full length antibody light chains each comprising in N- to C-terminal direction a light chain variable region and a light chain constant domain, as well as two full length antibody heavy chains each comprising in N- to C-terminal direction a heavy chain variable region, a first heavy chain constant domain, a hinge region, a second heavy chain constant domain and a third heavy chain constant domain. In contrast to a native antibody, a full length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full length antibody.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, δ, ε, γ, and μ, respectively.

The term “heavy chain constant region” denotes the region of an immunoglobulin heavy chain that contains the constant domains, i.e. the CH1 domain, the hinge region, the CH2 domain and the CH3 domain. In one embodiment, a human IgG constant region extends from Ala118 to the carboxyl-terminus of the heavy chain (numbering according to Kabat EU index). However, the C-terminal lysine (Lys447) of the constant region may or may not be present (numbering according to Kabat EU index). The term “heavy chain constant region” denotes a dimer comprising two heavy chain constant regions, which can be covalently linked to each other via the hinge region cysteine residues forming inter-chain disulfide bonds.

The term “light chain constant region” denotes the region of an immunoglobulin light chain that contains the constant domain, i.e. the CL domain.

The term “constant region” encompasses both the “heavy chain constant region” and the “light chain constant region”.

The term “heavy chain Fc-region” denotes the C-terminal region of an immunoglobulin heavy chain that contains at least a part of the hinge region (middle and lower hinge region), the CH2 domain and the CH3 domain. In one embodiment, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but in the C-terminal part identical thereto. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The constant region, more precisely the Fc-region, of an antibody (and the constant region likewise) is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1q binding and C3 activation, whereas IgG4 do not activate the complement system, do not bind C1q and do not activate C3. An “Fc-region of an antibody” is a term well known to the skilled artisan and defined on the basis of papain cleavage of antibodies.

### Splicing

The constant region amino acid sequences of the different human immunoglobulins are encoded by corresponding DNA sequences. In the genome these DNA sequences contain coding (exonic) and non-coding (intronic) sequences. After transcription of the DNA to the pre-mRNA the pre-mRNA also contains these intronic and exonic sequences. Prior to translation the non-coding intronic sequences are removed during mRNA processing by splicing them out of the primary mRNA transcript to generate the mature mRNA. The splicing of the primary mRNA is controlled by a donor splice site in combination with a properly spaced apart acceptor splice site. The donor splice site is located at the 5′ end and the acceptor splice site is located at the 3′ end of an intronic sequence.

The term “properly spaced apart” denotes that a donor splice site and an acceptor splice site in a nucleic acid are arranged in such a way that all required elements for the splicing process are available and are in an appropriate position to allow the splicing process to take place.

A donor splice site (5′ splice site) is a nucleic acid sequence motif representing the 5′ end of an intron.

An acceptor splice site (3′ splice site) is a nucleic acid sequence motif representing the 3′ end of an intron.

### Codon Optimization:

The generation of recombinant antibodies is based at least for the constant regions on naturally occurring wild-type or germline sequences. In order to prevent biological limitations of these DNA templates such as RNA instabilities, inefficient nuclear export, secondary structures or insufficient translation rates gene optimization using bioinformatic technologies and the subsequent de novo synthesis of genes based on the protein sequence are performed [10]. Thus, complex and time-consuming cloning steps can be circumvented and, as shown in recent years, translation rates can be increased by such adjustments of codon usage to the production system [10]. For codon optimizations, a high GC content, the avoidance of splice sites and the adaptation of codon usage to the production organism play a central role. There have already been established several methods that examine the use and frequency of specific codons. It is known that tRNAs encoding the same amino acid compete with each other. On the one hand, multivalent tRNAs recognizing more than one codon are more commonly available, and on the other hand, different tRNA species are expressed with varying frequency. As a result, codons encoded by more abundant tRNAs also occur more frequently in the coding sequence [11]. In Table 1 three possible methods for applying codon usage adaptation/optimization are shown.

TABLE 1
Illustration/comparison of methods
Codon distribution

| Codon | Frequency |
|---|---|
| AAA | 3/6 |
| BBB | 2/6 |
| CCC | 1/6 |

| Ocura [symbol] | high \| | Method 1 | Method 2 |
|---|---|---|---|
| 1 | AAA | AAA | AAA |
| 2 | AAA | AAA | BBB |
| 3 | AAA | AAA | AAA |
| 4 | AAA | BBB | BBB |
| 5 | AAA | BBB | AAA |
| 6 | AAA | CCC | CCC |

[symbol]

indicates data missing or illegible when filed

The codon usage of one method is aimed at making the entire tRNA pool available for translation, i.e. method 2. In contrast to the “high” method, which uses only the most abundant codon for translation to the amino acid, all available codons are used. Method 2 also takes into account the distribution of codons used in the codon usage of each organism compared to Method 1 and distributes the codons within the sequence [11].

### Splice Sites:

Almost all genes coding for proteins in eukaryotes are divided into exons and introns. Currently, 12 variants of exons are known [12]. Splicing is the excision of introns from pre-mRNA during transcription. Correct splicing is based on conserved consensus sequences on the 5′- and 3′-end, respectively, of the intron and the so-called branch-site. The branch site is approximately 20-50 nucleotides upstream of the 3′-end of the intron [12]. At the 5′-end of the intron is the donor splice site with the characteristic dinucleotide GT. At the 3′-end is the acceptor splice site located with the bases AG [13]. The pattern is called a canonical pattern of the splice site. Nevertheless, 3.7% of annotated splice sites do not follow this pattern [14]. Splice sites with GC-AG, GG-AG, GT-TG, GT-CG or CT-AG dinucleotides within the introns were also observed [14]. Some of these non-canonical splice sites may be involved in the expression of immunoglobulins [15]. According to Burset et al. are the dinucleotides GC-AG the most common non-canonical pattern of splice sites. In addition, the consensus sequence is strongly dependent on the GC content [12]. At high GC content, the donor consensus sequence at the 5′-end is described as AG/GTRAGT (SEQ ID NO: 02) rather than as AG/GTAAGT (SEQ ID NO: 03) at low GC content [12].

Introns are cut through the spliceosome. Those introns flanked by the canonical GT-AG pair are released from pre-mRNA by spliceosomes with subunits U1, U2, U4/U6, and U5 [16]. In addition, it is known that the eukaryotic genome has a variety of cryptic splice sites that adversely affect proper splicing. These differ from the consensus motif of the splice sites. Normally, these sites are inactive or rarely used by the cell machinery [17], [18]. Cryptic splice sites occur in both introns and exons. The goal of bioinformatic programs is to recognize the position of these cryptic splice sites. Many of these programs are informative, but are inferior to the complexity of nucleotide information due to the many possibilities [22].

### Specific Embodiments of the Method According to the Invention

For the production of a therapeutic or diagnostic antibody high expression yields are the aim. One option to achieve good expression rates is to optimize the codon usage of the encoding nucleic acids at first and adjust it to the codon usage of the cell intended to express the exogenous nucleic acid. This codon adaptation or optimization can be done based on different established protocols.

But during such a codon adaptation and optimization process, e.g. non-paired splices sites can be generated de novo. That is, during codon optimization a new donor splice site sequence is generated in the codon usage optimized nucleic acid. This is independent on the organization of the codon usage optimize nucleic acid, i.e. it is possible for both cDNA or genomically organized nucleic acids. It is in fact an unintended side-result of the codon usage optimization process. As such a new donor splice site is an additional artificial donor splice site it does not have an associated target acceptor splice site. Thus, such non-paired donor splice sites can give rise to splicing events with a random, i.e. non-defined, acceptor splice site present somewhere in the transcribed mRNA. Thereby the expression yield is reduced.

The invention is based, at least in part, on the unexpected finding that the removal of non-paired donor splice sites in a codon usage optimized antibody heavy chain encoding nucleic acid needs only to be performed in the part of the nucleic acid encoding the variable domain of the heavy chain but not in the part encoding the constant region, i.e. for the constant region, e.g., the germline or wild-type human nucleic acid sequence can be used.

The current invention is based, at least in part, on the finding that the introduction of amino acid sequence silent nucleotide changes (mutations) in the non-paired donor splice site consensus sequence NGGTA(G)AG (SEQ ID NO: 01) only in the codon optimized nucleic acid encoding the variable domain of an antibody heavy chain is sufficient to improve the expression yield or allow expression of said antibody at all.

The invention is based, at least in part, on the finding that the removal of non-paired donor splice sites needs only to be performed in the nucleic acid encoding the variable domain of the heavy chain but not in the constant region, i.e. for the constant region either the wild-type sequence, the germline sequence or a sequence optimized with standard methods, such as that reported in WO 2013/15644, can be used.

The invention is based, at least in part, on the finding that the expression yield can be increased by using the method according to the invention with the light chain.

The current invention is based on the usage of the donor consensus sequence of SEQ ID NO: 01: NGGTA(G)AG. This sequence has already been identified by Zhang et al. 1998, but found no entry into the codon optimization protocols.

The dinucleotide GT marks the beginning of an intron and thereby a splice site. The thereafter following base can be an adenine or a guanine.

Another parameter that could be taken into account is the number of allowed mismatches in this sequence, in order to adjust the sensitive and stringency of the method.

A consensus acceptor splice sequence has the sequence of SEQ ID NO: 04: [CT]n N [CT] AG with N=any base, n=number of CT dinucleotides. This sequence has also already been identified by Zhang 1998.

The method according to the current invention is exemplified in the following with specific antibodies. These examples shall not be understood as a limitation of the invention. These are presented as a mere exemplification of the generally applicable method according to the current invention.

In the following Table 2 the expression yields for differently processed antibody heavy and/or light chain encoding nucleic acids are summarized. The results have been obtained by transient expression in HEK293 cells using a two plasmid system.

Construct ‘00’ is the starting nucleic acid.

Constructs ‘00’-‘06’ and ‘16’ have been codon optimized with a method known in the art from WO 2013/156443=reference prior art method.

Constructs ‘07’-‘11’ and ‘17’ have been processed according to the current invention, i.e. the reference prior art method from WO 2013/156443 supplemented with the removal of donor splice site based on the consensus motif of SEQ ID NO: 01.

Constructs ‘12’-‘15’ have been codon optimized by a commercial provider, Geneart, using an approach different from WO 2013/156443 as second reference prior art method.

The term “hu” denotes that human codon usage was used, whereas the term “CHO” denotes that Chinese hamster codon usage was used.

TABLE 2

| | | heavy | heavy | | |
|---|---|---|---|---|---|
| | | chain | chain | | |
| | light | variable | constant | | yield |
| construct | chain | region | region | organization | [mg/L] |
| 00a | not | reference | IgG1-hu- | genomically | 4 |
| | optimized | prior art | wt | organized | |
| 00 | method | method | IgG1-hu- | genomically | 8 |
| | according | | wt | organized | |
| | to the | | | | |
| | invention | | | | |
| 01 | method | | IgG1- | genomically | 0 |
| | according | | CHO- | organized | |
| | to the | | method | | |
| | invention | | according | | |
| | | | to the | | |
| | | | invention | | |
| 16 | method | | IgG1-hu- | genomically | 0 |
| | according | | method | organized | |
| | to the | | according | | |
| | invention | | to the | | |
| | | | invention | | |
| 02a | not | | IgG1-hu- | cDNA | 123 |
| | optimized | | wt | | |
| 02 | method | | IgG1-hu- | cDNA | 120 |
| | according | | wt | | |
| | to the | | | | |
| | invention | | | | |
| 03a | not | | IgG1-hu- | cDNA | 92 |
| | optimized | | reference | | |
| | | | prior art | | |
| | | | method | | |
| 03 | method | | IgG1-hu- | cDNA | 126.5 |
| | according | | reference | | |
| | to the | | prior art | | |
| | invention | | method | | |
| 04a | not | | IgG1- | cDNA | 86 |
| | optimized | | CHO- | | |
| | | | reference | | |
| | | | prior art | | |
| | | | method | | |
| 04 | reference | | IgG1- | cDNA | 118.5 |
| | prior art | | CHO- | | |
| | method | | reference | | |
| | | | prior art | | |
| | | | method | | |
| 05a | not | | IgG1- | cDNA | 61 |
| | optimized | | CHO- | | |
| | | | method | | |
| | | | according | | |
| | | | to the | | |
| | | | invention | | |
| 05 | method | | IgG1- | cDNA | 114 |
| | according | | CHO- | | |
| | to the | | method | | |
| | invention | | according | | |
| | | | to the | | |
| | | | invention | | |
| 05a | not | | IgG1- | cDNA | 136 |
| | optimized | | CHO- | | |
| | | | Geneart | | |
| 06 | method | | IgG1- | cDNA | 130.5 |
| | according | | CHO- | | |
| | to the | | Geneart | | |
| | invention | | | | |
| 07 | method | method | IgG1-hu- | genomically | 91 |
| | according | according | wt | organized | |
| | to the | to the | | | |
| | invention | invention | | | |
| 17 | method | | IgG1-hu- | genomically | 65 |
| | according | | method | organized | |
| | to the | | according | | |
| | invention | | to the | | |
| | | | invention | | |
| 08 | method | | IgG1-wt | cDNA | 128 |
| | according | | | | |
| | to the | | | | |
| | invention | | | | |
| 09 | method | | IgG1-hu- | cDNA | 93 |
| | according | | reference | | |
| | to the | | prior art | | |
| | invention | | method | | |
| 10 | method | | IgG1- | cDNA | 92 |
| | according | | CHO- | | |
| | to the | | reference | | |
| | invention | | prior art | | |
| | | | method | | |
| 11 | method | | IgG1- | cDNA | 105 |
| | according | | CHO- | | |
| | to the | | method | | |
| | invention | | according | | |
| | | | to the | | |
| | | | invention | | |
| 12 | method | optimization | IgG1-wt | genomically | 78 |
| | according | by Geneart | | organized | |
| | to the | | | | |
| | invention | | | | |
| 13 | method | | IgG1-wt | cDNA | 79 |
| | according | | | | |
| | to the | | | | |
| | invention | | | | |
| 14 | method | | IgG1- | cDNA | 65 |
| | according | | CHO- | | |
| | to the | | reference | | |
| | invention | | prior art | | |
| | | | method | | |
| 15 | method | | IgG1- | cDNA | 50 |
| | according | | CHO- | | |
| | to the | | method | | |
| | invention | | according | | |
| | | | to the | | |
| | | | invention | | |

From the data the following can be seen:

for the light chain:
when using genomic organization, processing according to the current invention results in comparable expression yield,

| | | heavy | heavy | | |
|---|---|---|---|---|---|
| | | chain | chain | | |
| | light | variable | constant | | yield |
| construct | chain | region | region | organization | [mg/L] |
| 00a | not | reference | IgG1-hu- | genomically | 4 |
| | optimized | prior art | wt | organized | |
| 00 | method | method | IgG1-hu- | genomically | 8 |
| | according | | wt | organized | |
| | to the | | | | |
| | invention | | | | |

the use of cDNA results in general in increased expression yields,

| | | heavy | heavy | | |
|---|---|---|---|---|---|
| | | chain | chain | | |
| | light | variable | constant | | yield |
| construct | chain | region | region | organization | [mg/L] |
| 00a | not | reference | IgG1-hu- | genomically | 4 |
| | optimized | prior art | wt | organized | |
| 00 | method | method | IgG1-hu- | genomically | 8 |
| | according | | wt | organized | |
| | to the | | | | |
| | invention | | | | |
| 02a | not | | IgG1-hu- | cDNA | 123 |
| | optimized | | wt | | |
| 02 | method | | IgG1-hu- | cDNA | 120 |
| | according | | wt | | |
| | to the | | | | |
| | invention | | | | |

when using cDNA, the processing according to the current invention results in most cases in increased expression yield, in the other cases the expression yield is comparable,

| | | heavy | heavy | | |
|---|---|---|---|---|---|
| | | chain | chain | | |
| | light | variable | constant | | yield |
| construct | chain | region | region | organization | [mg/L] |
| 02a | not | reference | IgG1-hu- | cDNA | 123 |
| | optimized | prior art | wt | | |
| 02 | method | method | IgG1-hu- | cDNA | 120 |
| | according | | wt | | |
| | to the | | | | |
| | invention | | | | |
| 03a | not | | IgG1-hu- | cDNA | 92 |
| | optimized | | reference | | |
| | | | prior art | | |
| | | | method | | |
| 03 | method | | IgG1-hu- | cDNA | 126.5 |
| | according | | reference | | |
| | to the | | prior art | | |
| | invention | | method | | |
| 04a | not | | IgG1- | cDNA | 86 |
| | optimized | | CHO- | | |
| | | | reference | | |
| | | | prior art | | |
| | | | method | | |
| 04 | reference | | IgG1- | cDNA | 118.5 |
| | prior art | | CHO- | | |
| | method | | reference | | |
| | | | prior art | | |
| | | | method | | |
| 05a | not | | IgG1- | cDNA | 61 |
| | optimized | | CHO- | | |
| | | | method | | |
| | | | according | | |
| | | | to the | | |
| | | | invention | | |
| 05 | method | | IgG1- | cDNA | 114 |
| | according | | CHO- | | |
| | to the | | method | | |
| | invention | | according | | |
| | | | to the | | |
| | | | invention | | |
| 05a | not | | IgG1- | cDNA | 136 |
| | optimized | | CHO- | | |
| | | | Geneart | | |
| 06 | method | | IgG1- | cDNA | 130.5 |
| | according | | CHO- | | |
| | to the | | Geneart | | |
| | invention | | | | |

for the heavy chain:
when using genomic organization, the processing according to the current invention results in increased expression yield or in expression at all, if at least the heavy chain variable domain is optimized,

| | | heavy | heavy | | |
|---|---|---|---|---|---|
| | | chain | chain | | |
| | light | variable | constant | | yield |
| construct | chain | region | region | organization | [mg/L] |
| 00 | method | reference | IgG1-hu- | genomically | 8 |
| | according | prior art | wt | organized | |
| | to the | method | | | |
| | invention | | | | |
| 16 | method | | IgG1-hu- | genomically | 0 |
| | according | | method | organized | |
| | to the | | according | | |
| | invention | | to the | | |
| | | | invention | | |
| 07 | method | method | IgG1-hu- | genomically | 91 |
| | according | according | wt | organized | |
| | to the | to the | | | |
| | invention | invention | | | |
| 17 | method | | IgG1-hu- | genomically | 65 |
| | according | | method | organized | |
| | to the | | according | | |
| | invention | | to the | | |
| | | | invention | | |
| 12 | Method | Optimization | IgG1-wt | Genomically | 78 |
| | according | by Geneart | | organized | |
| | to the | | | | |
| | invention | | | | |

the use of cDNA results in general in increased expression yields.

| | | heavy | heavy | | |
|---|---|---|---|---|---|
| | | chain | chain | | |
| | light | variable | constant | | yield |
| construct | chain | region | region | organization | [mg/L] |
| 00 | method | reference | IgG1-hu- | genomically | 8 |
| | according | prior art | wt | organized | |
| | to the | method | | | |
| | invention | | | | |
| 16 | method | | IgG1-hu- | genomically | 0 |
| | according | | method | organized | |
| | to the | | according | | |
| | invention | | to the | | |
| | | | invention | | |
| 02 | method | | IgG1-hu- | cDNA | 120 |
| | according | | wt | | |
| | to the | | | | |
| | invention | | | | |
| 03 | method | | IgG1-hu- | cDNA | 126.5 |
| | according | | reference | | |
| | to the | | prior art | | |
| | invention | | method | | |
| 04 | method | | IgG1- | cDNA | 118.5 |
| | according | | CHO- | | |
| | to the | | reference | | |
| | invention | | prior art | | |
| | | | method | | |
| 05 | method | | IgG1- | cDNA | 114 |
| | according | | CHO- | | |
| | to the | | method | | |
| | invention | | according | | |
| | | | to the | | |
| | | | invention | | |
| 06 | method | | IgG1- | cDNA | 130.5 |
| | according | | CHO- | | |
| | to the | | Geneart | | |
| | invention | | | | |
| 07 | method | method | IgG1-hu- | genomically | 91 |
| | according | according | wt | organized | |
| | to the | to the | | | |
| | invention | invention | | | |
| 17 | method | | IgG1-hu- | genomically | 65 |
| | according | | method | organized | |
| | to the | | according | | |
| | invention | | to the | | |
| | | | invention | | |
| 08 | method | | IgG1-wt | cDNA | 128 |
| | according | | | | |
| | to the | | | | |
| | invention | | | | |
| 09 | method | | IgG1-hu- | cDNA | 93 |
| | according | | reference | | |
| | to the | | prior art | | |
| | invention | | method | | |
| 10 | method | | IgG1- | cDNA | 92 |
| | according | | CHO- | | |
| | to the | | reference | | |
| | invention | | prior art | | |
| | | | method | | |
| 11 | method | | IgG1- | cDNA | 105 |
| | according | | CHO- | | |
| | to the | | method | | |
| | invention | | according | | |
| | | | to the | | |
| | | | invention | | |

when using cDNA, the processing according to the current invention also to the constant region of the heavy chain seems to not further increase the expression yield,
when using cDNA, the processing according to the current invention results in a further increased expression yield,

| | | heavy | heavy | | |
|---|---|---|---|---|---|
| | | chain | chain | | |
| | light | variable | constant | | yield |
| construct | chain | region | region | organization | [mg/L] |
| 02 | method | reference | IgG1-hu- | cDNA | 120 |
| | according | prior art | wt | | |
| | to the | method | | | |
| | invention | | | | |
| 08 | method | method | IgG1-wt | cDNA | 128 |
| | according | according | | | |
| | to the | to the | | | |
| | invention | invention | | | |

Thus, one aspect as reported herein is a method for producing an immunoglobulin comprising the following steps:
cultivating a mammalian cell, preferably a CHO cell, comprising a nucleic acid with intron-exon organization encoding an immunoglobulin heavy chain of the human IgG1 subclass and an immunoglobulin light chain, so that the immunoglobulin is expressed,
recovering the immunoglobulin from the cell or the cultivation medium and thereby producing the immunoglobulin,
   wherein in the part of the nucleic acid encoding the immunoglobulin heavy chain variable domain, non-paired donor splice sites according to SEQ ID NO: 01 are removed by introducing amino acid sequence silent nucleotide changes in the non-paired donor splice site consensus sequence NGGTA(G)AG (SEQ ID NO: 01) in the codon NGG or the codon GGT or the codon GTA(G).

### Reference Codon Optimization Method

The nucleic acid encoding an immunoglobulin can be optimized, e.g., by adapting the general codon usage according to the method as reported in WO 2013/156443.

Said reference method is based on the finding that for the expression of a polypeptide in a cell the use of a polypeptide encoding nucleic acid that is characterized in that each amino acid is encoded by a group of codons, whereby each codon in the group of codons is defined by a specific usage frequency within the group that is related to the overall usage frequency of this codon in the genome of the cell, and whereby the usage frequency of the codons in the (total) polypeptide encoding nucleic acid is about the same as the usage frequency within the respective group.

The reference method is a method for recombinantly producing a polypeptide in a mammalian cell comprising the step of cultivating a cell which comprises a nucleic acid encoding the polypeptide, and recovering the polypeptide from the mammalian cell or the cultivation medium,
wherein each of the amino acid residues of the polypeptide is encoded by one or more (at least one) codon(s), whereby the (different) codons encoding the same amino acid residue are combined in one group and each of the codons in a group is defined by a specific usage frequency within the group, which is the frequency with which a single codon of a group of codons can be found in a nucleic acid encoding a polypeptide in relation to all codons of one group, whereby the sum of the specific usage frequencies of all codons in one group is 100%,
wherein the overall usage frequency of each codon in the polypeptide encoding nucleic acid is about the same as its specific usage frequency within its group.

In one embodiment the amino acid residues G, A, V, L, I, P, F, S, T, N, Q, Y, C, K, R, H, D, and E are each encoded by a group of codons and the amino acid residues M and W are encoded by a single codon.

In one embodiment the amino acid residues G, A, V, L, I, P, F, S, T, N, Q, Y, C, K, R, H, D, and E are each encoded by a group of codons comprising at least two codons and the amino acid residues M and W are encoded by a single codon.

In one embodiment the specific usage frequency of a codon is 100% if the amino acid residue is encoded by exactly one codon.

In one embodiment the amino acid residue G is encoded by a group of at most 4 codons. In one embodiment the amino acid residue A is encoded by a group of at most 4 codons. In one embodiment the amino acid residue V is encoded by a group of at most 4 codons. In one embodiment the amino acid residue L is encoded by a group of at most 6 codons. In one embodiment the amino acid residue I is encoded by a group of at most 3 codons. In one embodiment the amino acid residue M is encoded by exactly 1 codon. In one embodiment the amino acid residue P is encoded by a group of at most 4 codons. In one embodiment the amino acid residue F is encoded by a group of at most 2 codons. In one embodiment the amino acid residue W is encoded by exactly 1 codon. In one embodiment the amino acid residue S is encoded by a group of at most 6 codons. In one embodiment the amino acid residue T is encoded by a group of at most 4 codons. In one embodiment the amino acid residue N is encoded by a group of at most 2 codons. In one embodiment the amino acid residue Q is encoded by a group of at most 2 codons. In one embodiment the amino acid residue Y is encoded by a group of at most 2 codons. In one embodiment the amino acid residue C is encoded by a group of at most 2 codons. In one embodiment the amino acid residue K is encoded by a group of at most 2 codons. In one embodiment the amino acid residue R is encoded by a group of at most 6 codons. In one embodiment the amino acid residue H is encoded by a group of at most 2 codons. In one embodiment the amino acid residue D is encoded by a group of at most 2 codons. In one embodiment the amino acid residue E is encoded by a group of at most 2 codons.

In one embodiment the amino acid residue G is encoded by a group of 1 to 4 codons. In one embodiment the amino acid residue A is encoded by a group of 1 to 4 codons. In one embodiment the amino acid residue V is encoded by a group of 1 to 4 codons. In one embodiment the amino acid residue L is encoded by a group of 1 to 6 codons. In one embodiment the amino acid residue I is encoded by a group of 1 to 3 codons. In one embodiment the amino acid residue M is encoded by a group of 1 codon, i.e. by exactly 1 codon. In one embodiment the amino acid residue P is encoded by a group of 1 to 4 codons. In one embodiment the amino acid residue F is encoded by a group of 1 to 2 codons. In one embodiment the amino acid residue W is encoded by a group of 1 codon, i.e. by exactly 1 codon. In one embodiment the amino acid residue S is encoded by a group of 1 to 6 codons. In one embodiment the amino acid residue T is encoded by a group of 1 to 4 codons. In one embodiment the amino acid residue N is encoded by a group of 1 to 2 codons. In one embodiment the amino acid residue Q is encoded by a group of 1 to 2 codons. In one embodiment the amino acid residue Y is encoded by a group of 1 to 2 codons. In one embodiment the amino acid residue C is encoded by a group of 1 to 2 codons. In one embodiment the amino acid residue K is encoded by a group of 1 to 2 codons. In one embodiment the amino acid residue R is encoded by a group of 1 to 6 codons. In one embodiment the amino acid residue H is encoded by a group of 1 to 2 codons. In one embodiment the amino acid residue D is encoded by a group of 1 to 2 codons. In one embodiment the amino acid residue E is encoded by a group of 1 to 2 codons.

In one embodiment each of the groups comprises only codons with an overall usage frequency within the genome of the cell of more than 5%. In one embodiment each of the groups comprises only codons with an overall usage frequency within the genome of the cell of 8% or more. In one embodiment each of the groups comprises only codons with an overall usage frequency within the genome of the cell of 10% or more. In one embodiment each of the groups comprises only codons with an overall usage frequency within the genome of the cell of 15% or more.

In one embodiment the sequence of codons in the nucleic acid encoding the polypeptide for a specific amino acid residue in 5′ to 3′ direction is, i.e. corresponds to, the sequence of codons in a respective amino acid codon motif.

In one embodiment for each sequential occurrence of a specific amino acid in the polypeptide starting from the N-terminus of the polypeptide the encoding nucleic acid comprises the codon that is the same as that at the corresponding sequential position in the amino acid codon motif of the respective specific amino acid, whereby upon the first occurrence of the amino acid residue in the amino acid sequence of the polypeptide the first codon of the amino acid codon motif is used in the corresponding encoding nucleic acid, upon the second occurrence of the amino acid residue the second codon of the amino acid codon motif is used and so on.

In one embodiment the usage frequency of a codon in the amino acid codon motif is about the same as its specific usage frequency within its group.

In one embodiment after the final codon of the amino acid codon motif has been reached at the next occurrence of the specific amino acid in the polypeptide the encoding nucleic acid comprises the codon that is at the first position of the amino acid codon motif.

In one embodiment the codons in the amino acid codon motif are distributed randomly throughout the amino acid codon motif.

In one embodiment the amino acid codon motif is selected from a group of amino acid codon motifs comprising all possible amino acid codon motifs obtainable by permutating codons therein wherein all motifs have the same number of codons and the codons in each motif have the same specific usage frequency.

In one embodiment the codons in the amino acid codon motif are arranged with decreasing specific usage frequency whereby all codons of one usage frequency directly succeed each other. In one embodiment the codons of one codon usage frequence are grouped together.

In one embodiment the (different) codons in the amino acid codon motif are distributed uniformly throughout the amino acid codon motif.

In one embodiment the codons in the amino acid codon motif are arranged with decreasing specific usage frequency whereby after the codon with the lowest specific usage frequency or the codon with the second lowest specific usage frequency the codon with the highest specific usage frequency is present (used).

In one embodiment the codons in the amino acid codon motif are arranged with decreasing specific usage frequency whereby after the codon with the lowest specific usage frequency the codon with the highest specific usage frequency is present (used).

Thus, the nucleic acid encoding a polypeptide is characterized in that each of the amino acid residues of the polypeptide is encoded by one or more (at least one) codon(s),
whereby the different codons encoding the same amino acid residue are combined in one group and each of the codons in a group is defined by a specific usage frequency within the group, which is the frequency with which a single codon of a group of codons can be found in a nucleic acid encoding a polypeptide in relation to all codons of one group, whereby the sum of the specific usage frequencies of all codons in one group is 100%,
wherein the usage frequency of a codon in the polypeptide encoding nucleic acid is about the same as its specific usage frequency within its group.

In one embodiment the amino acid residues G, A, V, L, I, P, F, S, T, N, Q, Y, C, K, R, H, D, and E are each encoded by a group of codons and the amino acid residues M and W are encoded by a single codon.

In one embodiment the amino acid residues G, A, V, L, I, P, F, S, T, N, Q, Y, C, K, R, H, D, and E are each encoded by a group of codons comprising at least two codons and the amino acid residues M and W are encoded by a single codon.

In one embodiment the specific usage frequency of a codon is 100% if the amino acid residue is encoded by exactly one codon.

In one embodiment the amino acid residue G is encoded by a group of at most 4 codons. In one embodiment the amino acid residue A is encoded by a group of at most 4 codons. In one embodiment the amino acid residue V is encoded by a group of at most 4 codons. In one embodiment the amino acid residue L is encoded by a group of at most 6 codons. In one embodiment the amino acid residue I is encoded by a group of at most 3 codons. In one embodiment the amino acid residue M is encoded by exactly 1 codon. In one embodiment the amino acid residue P is encoded by a group of at most 4 codons. In one embodiment the amino acid residue F is encoded by a group of at most 2 codons. In one embodiment the amino acid residue W is encoded by exactly 1 codon. In one embodiment the amino acid residue S is encoded by a group of at most 6 codons. In one embodiment the amino acid residue T is encoded by a group of at most 4 codons. In one embodiment the amino acid residue N is encoded by a group of at most 2 codons. In one embodiment the amino acid residue Q is encoded by a group of at most 2 codons. In one embodiment the amino acid residue Y is encoded by a group of at most 2 codons. In one embodiment the amino acid residue C is encoded by a group of at most 2 codons. In one embodiment the amino acid residue K is encoded by a group of at most 2 codons. In one embodiment the amino acid residue R is encoded by a group of at most 6 codons. In one embodiment the amino acid residue H is encoded by a group of at most 2 codons. In one embodiment the amino acid residue D is encoded by a group of at most 2 codons. In one embodiment the amino acid residue E is encoded by a group of at most 2 codons.

In one embodiment the amino acid residue G is encoded by a group of 1 to 4 codons. In one embodiment the amino acid residue A is encoded by a group of 1 to 4 codons. In one embodiment the amino acid residue V is encoded by a group of 1 to 4 codons. In one embodiment the amino acid residue L is encoded by a group of 1 to 6 codons. In one embodiment the amino acid residue I is encoded by a group of 1 to 3 codons. In one embodiment the amino acid residue M is encoded by a group of 1 codon. In one embodiment the amino acid residue P is encoded by a group of 1 to 4 codons. In one embodiment the amino acid residue F is encoded by a group of 1 to 2 codons. In one embodiment the amino acid residue W is encoded by a group of 1 codon. In one embodiment the amino acid residue S is encoded by a group of 1 to 6 codons. In one embodiment the amino acid residue T is encoded by a group of 1 to 4 codons. In one embodiment the amino acid residue N is encoded by a group of 1 to 2 codons. In one embodiment the amino acid residue Q is encoded by a group of 1 to 2 codons. In one embodiment the amino acid residue Y is encoded by a group of 1 to 2 codons. In one embodiment the amino acid residue C is encoded by a group of 1 to 2 codons. In one embodiment the amino acid residue K is encoded by a group of 1 to 2 codons. In one embodiment the amino acid residue R is encoded by a group of 1 to 6 codons. In one embodiment the amino acid residue H is encoded by a group of 1 to 2 codons. In one embodiment the amino acid residue D is encoded by a group of 1 to 2 codons. In one embodiment the amino acid residue E is encoded by a group of 1 to 2 codons.

In one embodiment each of the groups comprises only codons with an overall usage frequency within the genome of the cell of more than 5%. In one embodiment each of the groups comprises only codons with an overall usage frequency within the genome of the cell of 8% or more. In one embodiment each of the groups comprises only codons with an overall usage frequency within the genome of the cell of 10% or more. In one embodiment each of the groups comprises only codons with an overall usage frequency within the genome of the cell of 15% or more.

In one embodiment the sequence of codons in the nucleic acid encoding the polypeptide for a specific amino acid residue in 5′ to 3′ direction is, i.e. corresponds to, the sequence of codons in a respective amino acid codon motif.

In one embodiment for each sequential occurrence of a specific amino acid in the polypeptide starting from the N-terminus of the polypeptide the encoding nucleic acid comprises the codon that is the same as that at the corresponding sequential position in the amino acid codon motif of the respective specific amino acid, whereby upon the first occurrence of the amino acid residue in the amino acid sequence of the polypeptide the first codon of the amino acid codon motif is used in the corresponding encoding nucleic acid, upon the second occurrence of the amino acid residue the second codon of the amino acid codon motif is used and so on.

In one embodiment the usage frequency of a codon in the amino acid codon motif is about the same as its specific usage frequency within its group.

In one embodiment after the final codon of the amino acid codon motif has been reached at the next occurrence of the specific amino acid in the polypeptide the encoding nucleic acid comprises the codon that is at the first position of the amino acid codon motif.

In one embodiment each of the codons in the amino acid codon motif is distributed randomly throughout the amino acid codon motif.

In one embodiment each of the codons in the amino acid codon motif is distributed evenly throughout the amino acid codon motif.

In one embodiment the codons in the amino acid codon motif are arranged with decreasing specific usage frequency whereby after the codon with the lowest specific usage frequency or the codon with the second lowest specific usage frequency the codon with the highest specific usage frequency is used.

In one embodiment the codons in the amino acid codon motif are arranged with decreasing specific usage frequency whereby after the codon with the lowest specific usage frequency the codon with the highest specific usage frequency is used.

Thus, the method according to the current invention is a method for increasing the expression of a polypeptide in a eukaryotic cell comprising the step of,
providing a nucleic acid encoding the polypeptide,
wherein each of the amino acid residues of the polypeptide is encoded by at least one codon, whereby the different codons encoding the same amino acid residue are combined in one group and each of the codons in a group is defined by a specific usage frequency within the group, whereby the sum of the specific usage frequencies of all codons in one group is 100%,
wherein the usage frequency of a codon in the polypeptide encoding nucleic acid is about the same as its specific usage frequency within its group,
wherein donor splice site according to the consensus sequence of SEQ ID NO: 01 are removed from the nucleic acid encoding the heavy chain variable domain.

### Recombinant Methods

Antibodies may be produced using recombinant methods and compositions, e.g., as described in U.S. Pat. No. 4,816,567. The antibody encoding nucleic acids may encode an amino acid sequence comprising the VL and/or an amino acid sequence comprising the VH of the antibody (e.g., the light and/or heavy chains of the antibody). In one embodiment, the cell expressing the immunoglobulin constant region containing polypeptide has been transfected with one or more vectors (e.g., expression vectors) comprising such nucleic acid. In one embodiment, a cell comprising such nucleic acid modified with the method as reported herein is provided. In one embodiment, a cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the cell is eukaryotic, e.g. a Chinese Hamster Ovary (CHO) cell or lymphoid cell (e.g., YO, NSO, Sp2/0 cell). In one embodiment, a method of making an antibody is provided, wherein the method comprises culturing a cell comprising a nucleic acid encoding the antibody, as provided herein, under conditions suitable for expression of the antibody, and optionally recovering the antibody from the cell (or culture medium).

For recombinant production of an antibody, nucleic acid encoding an antibody, e.g., as reported herein, are generated and inserted into one or more vectors for further cloning and/or expression in a cell. Such nucleic acid may be readily isolated and sequenced using conventional procedures (e.g., by using oligonucleotide probes that are capable of binding specifically to genes encoding the heavy and light chains of the antibody).

Suitable cells for cloning or expression of antibody-encoding vectors include eukaryotic cells described herein.

In addition, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (see Gerngross, T. U., Nat. Biotech. 22 (2004) 1409-1414; and Li, H., et al., Nat. Biotech. 24 (2006) 210-215).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (see e.g. U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian cell lines are monkey kidney CV1 line transformed by SV40 (COS-7); human embryonic kidney line (HEK293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian cell lines include Chinese hamster ovary (CHO) cells, including DHFR⁻ CHO cells (Urlaub, G., et al., Proc. Natl. Acad. Sci. USA 77 (1980) 4216-4220); and myeloma cell lines such as YO, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004) pp. 255-268.

### Purification

Different methods are well established and widespread used for protein recovery and purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A. Appl. Biochem. Biotech. 75 (1998) 93-102).

### Codon Usage

Codon usage tables (see tables above for examples) are readily available, for example, at the “Codon Usage Database” available at http://www.kazusa.or.jp/codon/and these tables can be adapted in a number of ways (Nakamura, Y., et al., Nucl. Acids Res. 28 (2000) 292).

For high yield expression of recombinant polypeptides, the encoding nucleic acid plays an important role. Naturally occurring and from nature isolated encoding nucleic acids are generally not optimized for high yield expression, especially if expressed in a heterologous host cell. Due to the degeneration of the genetic code one amino acid residue can be encoded by more than one nucleotide triplet (codon) except for the amino acids tryptophan and methionine. Thus, for one amino acid sequence different encoding codons (=corresponding encoding nucleic acid sequences) are possible.

The different codons encoding one amino acid residue are employed by different organisms with different relative frequency (codon usage). Generally, one specific codon is used with higher frequency than the other possible codons.

In WO 2001/088141 a reading frame optimization according to codon usage found in highly expressed mammalian genes is reported. For that purpose, a matrix was generated considering almost exclusively those codons that are used most frequently and, less preferably, those that are used second most frequently in highly expressed mammalian genes as depicted in the following table. Using these codons from highly expressed human genes a fully synthetic reading frame not occurring in nature was created, which, however encodes the very same product as the original wild-type gene construct.

In U.S. Pat. No. 8,128,938 different methods of codon optimization using the usage frequency of individual codons are reported, such as uniform optimization, full-optimization and minimal optimization.

In the following table the most frequently used codon (codon 1) and second most frequently used codon (codon 2) found in highly expressed mammalian genes is shown.

TABLE

| amino acid | codon 1 | codon 2 |
|---|---|---|
| Ala | GCC | GCT |
| Arg | AGG | AGA |
| Asn | AAC | AAT |
| Asp | GAC | GAT |
| Cys | TGC | TGT |
| End | TGA | TAA |
| Gln | CAG | CAT |
| Glu | GAG | GAA |
| Gly | GGC | GGA |
| His | CAC | CAT |
| Ile | ATC | ATT |
| Leu | CTG | CTC |
| Lys | AAG | AAT |
| Met | ATG | ATG |
| Phe | TTC | TTT |
| Pro | CCC | CCT |
| Ser | AGC | TCC |
| Thr | ACC | ACA |
| Trp | TGG | TGG |
| Tyr | TAC | TAT |
| Val | GTG | GTC |

(Ausubel, F. M., et al., Current Protocols in Molecular Biology 2 (1994), A1.8-A1.9).

Few deviations from strict adherence to the usage of most frequently found codons may be made (i) to accommodate the introduction or removal of unique restriction sites, (ii) to break G or C stretches extending more than 7 base pairs in order to allow consecutive PCR amplification and sequencing of the synthetic gene product.

The following examples are provided to aid the understanding of the present invention, the true scope of which is set forth in the appended claims. It is understood that modifications can be made in the procedures set forth without departing from the spirit of the invention.

### LITERATURE

[10] M. Graf, L. Deml, and R. Wagner, “Codon-optimized genes that enable increased heterologous expression in mammalian cells and elicit efficient immune responses in mice after vaccination of naked DNA.,” Methods Mol. Med., vol. 94, pp. 197-210, 2004.
[11] WO 2013/156443.
[12] M. Q. Zhang, “Statistical features of human exons and their flanking regions,” vol. 7, no. 5, pp. 919-932, 1998.
[13] R. Breathnach, C. Benoist, K. O'Hare, F. Gannon, and P. Chambon, “Ovalbumin gene: evidence for a leader sequence in mRNA and DNA sequences at the exon-intron boundaries.,” Proc. Natl. Acad. Sci. U.S.A., vol. 75, no. 10, pp. 4853-4857, 1978.
[14] M. Burset, I. A. Seledtsov, and V. V Solovyev, “Analysis of canonical and non-canonical splice sites in mammalian genomes.,” Nucleic Acids Res., vol. 28, no. 21, pp. 4364-4375, 2000.
[15] M. B. Shapiro and P. Senapathy, “RNA splice junctions of different classes of eukaryotes: Sequence statistics and functional implications in gene expression,” Nucleic Acids Res., vol. 15, no. 17, pp. 7155-7174, 1987.
[16] T. W. Nilsen, “Twenty years of RNA: then and now,” pp. 471-473, 2015.
[17] R. A. Padgetr, P. J. Grabowski, M. M. Konarska, S. Seiler, and P. A. Sharp, “Splicing of messenger RNA precursors,” 1986.
[18] M. R. Green, “Pre-mRNA splicing,” Annu. Rev. Genet., vol. 20, pp. 671-708, 1986.
[22] Y. Kapustin, E. Chan, R. Sarkar, F. Wong, I. Vorechovsky, R. M. Winston, T. Tatusova, and N. J. Dibb, “Cryptic splice sites and split genes,” Nucleic Acids Res., vol. 39, no. 14, pp. 5837-5844, 2011.

### EXAMPLES

### Protein Determination:

The protein concentration was determined by determining the optical density (OD) at 280 nm, using the molar extinction coefficient calculated on the basis of the amino acid sequence.

### Recombinant DNA Technique:

Standard methods were used to manipulate DNA as described in Sambrook, J., et al., Molecular Cloning: A Laboratory Manual; Cold Spring Harbor Laboratory Press, Cold Spring Harbor, N.Y. (1989). The molecular biological reagents were used according to the manufacturer's instructions.

### Example 1

Expression and Purification of Antibodies from Differently Codon Optimized Nucleic Acids

The different not optimized or codon optimized variable domains were combined with wild-type human constant region encoding nucleic acids or with CHO codon usage optimized nucleic acids encoding human constant regions.

### Expression Plasmids:

Expression plasmids each comprise one expression cassette for the expression of the heavy or light chain. These were separately assembled in mammalian cell expression vectors.

General information regarding the nucleotide sequences of human light and heavy chains from which the codon usage can be deduced is given in: Kabat, E. A., et al., Sequences of Proteins of Immunological Interest, 5th ed., Public Health Service, National Institutes of Health, Bethesda, Md. (1991), NIH Publication No 91-3242.

Beside the light chain or heavy chain expression cassette these plasmids contain
a hygromycin resistance gene,
an origin of replication, oriP, of Epstein-Barr virus (EBV),
an origin of replication from the vector pUC18 which allows replication of this plasmid in E. coli, and
a β-lactamase gene which confers ampicillin resistance in E. coli.

### Recombinant DNA Techniques:

Cloning was performed using standard cloning techniques as described in Sambrook, J., et al., Molecular Cloning: A Laboratory Manual, second edition, Cold Spring Harbor Laboratory Press (1989). All molecular biological reagents were commercially available (if not indicated otherwise) and were used according to the manufacturer's instructions.

### DNA and Protein Sequence Analysis and Sequence Data Management:

The Vector NTI Advance suite version 9.0 was used for sequence creation, mapping, analysis, annotation, and illustration.

### Expression of Antibodies:

For the expression of the antibodies, human embryonic kidney cells (HEK) 293F were used. HEK293-cells are mainly used for transient gene expression. The desired protein can be harvested after a few days. HEK293F were cultivated in serum- and protein-free FreeStyle™ 293 expression medium (Gibco, Invitrogen™, Life Technologies) supplemented with penicillin and streptomycin (PenStrep) at 7% CO₂, 85% humidity and 37° C. in a shake flask. The cells were passaged every 3-4 days and split according to confluency. A cell count of 3×10⁵ cells/mL was always set. The cell count and vitality were measured with the CASEY Cell Counter (Roche), with 50 μl of the culture suspended in 10 ml Casyton and measured with the appropriate program.

For the transient transfection, the cell number was adjusted to 2×10⁶ cells/mL on the same day. The number of transient transfections should not be below passage 4 and not above passage 22. For the transfection, the transfection reagent PElpro was used and run for 7 days in the Fed Batch process. For transfection, the following volumes and DNA quantities were used for the transfection mix (culture volume 20 mL):

| | |
|---|---|
| volume/mL | 20 |
| F17 medium volume/mL | 0.8 |
| DNA/μg | 10 |
| PEIpro | 25.0 |
| F17 for VPA pre-dilution/mL | 4 |
| VPA 500 mM Stock/mL→ pre- | 0.2 |
| dilution 24 nM | |
| 0.6% Glucose/mL | 0.144 |
| 12% Feed 7/mL | 2.9 |

For the transfection the appropriate amounts of F17, DNA and transfection reagent were combined in the respective order, mixed and incubated for 10 minutes. Subsequently, the transfection mix was added to the cells. After about 3-5 hours, the corresponding pre-dilution of VPA was added. 16 hours later, the culture was supplemented with 0.6% glucose and 12% feed and further incubated.

| | |
|---|---|
| cultivation volume/mL | 30 |
| volume Opti-MEM/mL | 1.5 |
| DNA/μg | 30 |
| Opti-MEM for Fectin/mL | 1.5 |
| Enhancer 1/mL | 0.15 |
| Enhancer 2/mL | 1.5 |
| Glucose-45%/mL | 0.2 |
| ExpiFectamine293 | 80 μL |

After one week, the supernatant could be harvested. For this purpose, the mixture was centrifuged for 20 min at 1200 rpm and the supernatant filtered off sterile with a 0.22 μm filter.

### Expression Yield Determination:

Quantitation of the expressed antibodies from the supernatant was done with an HPLC column packed with protein A. Protein A is a cell wall-associated protein from Staphylococcus aureus that specifically binds to the constant region of IgG antibodies. Protein A is immobilized on a polymeric carrier and then binds to the target molecule. By altering various parameters such as pH and temperature, the antibody can be eluted and detected.

Seven days after transfection the HEK 293 cell supernatants were harvested. The recombinant antibody contained therein were purified from the supernatant by affinity chromatography using protein A-Sepharose™ affinity chromatography (GE Healthcare, Sweden). Briefly, the antibody containing clarified culture supernatants were applied on a MabSelectSuRe Protein A (5-50 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. The antibodies (or -derivatives) were eluted with 50 mM citrate buffer, pH 3.2. The protein containing fractions were neutralized with 0.1 ml 2 M Tris buffer, pH 9.0.

1. A method for producing an antibody of the human IgG1 subclass by cultivating a CHO cell that has been transfected with one or more expression cassettes comprising the nucleic acids encoding the heavy and the light chains of the antibody,
wherein the nucleic acids encoding the antibody heavy and light chains are codon usage optimized with the codon usage of human cells and/or the codon usage of CHO cells,
wherein in the part of the nucleic acid encoding the heavy chain variable domain at least one non-paired donor splice site is removed.

2. The method according to claim 1, wherein in the part of the nucleic acid encoding the heavy chain constant region non-paired donor splice sites are not removed.

3. The method according to claim 1, wherein in the nucleic acid encoding the antibody light chain non-paired splice sites are removed.

4. The method according to claim 1, wherein the transfection is a transient transfection.

5. The method according to claim 1, wherein the nucleic acids encoding the antibody are cDNA.

6. The method according to claim 1, wherein the nucleic acids encoding the light or/and the heavy chain of the antibody are genomically organized DNA.

7. The method according to claim 3, wherein the removal of non-paired splice sites is by removal of non-paired donor splice sites by introducing amino acid sequence silent nucleotide changes in the non-paired donor splice site nucleic acid sequence.

8. The method according to claim 3, wherein the removal of the non-paired splice sites is by introducing an amino acid silent mutation in the nucleotide sequence NGGTA(G)AG (SEQ ID NO: 01).

9. The method according to claim 8, wherein the removal of the non-paired splice sites is by introducing an amino acid silent mutation in the nucleotide sequence NGGTA(G)AG (SEQ ID NO: 01) in the codon NGG or the codon GGT or the codon GTA(G).

10. The method according to claim 1, wherein the method comprises the following steps:
a) cultivating the CHO cell, and
b) recovering the antibody from the CHO cell or the cultivation medium.

11. A method for producing an antibody of the human IgG1 subclass by cultivating a CHO cell comprising one or more nucleic acids encoding the heavy and the light chains of the antibody,
wherein in the nucleic acid encoding the heavy chain variable domain non-paired donor splice sites are removed by introducing amino acid sequence silent nucleotide changes in the non-paired donor splice site consensus sequence NGGTA(G)AG (SEQ ID NO: 01) in the codon NGG or the codon GGT or the codon GTA(G),
wherein non-paired donor splice sites according to the sequence of SEQ ID NO: 01 are not removed in the nucleic acid encoding the heavy chain constant region.

12. The method according to claim 11, wherein the one or more nucleic acids encoding the antibody heavy and light chains are genomically organized DNA.

13. The method according to claim 12, wherein the one or more nucleic acids encoding the antibody heavy and light chains are codon usage optimized with the codon usage of human cells and/or the codon usage of CHO cells.

14. The method according to claim 13, wherein in the nucleic acid encoding the antibody light chain non-paired splice sites are removed by introducing amino acid sequence silent nucleotide changes in the non-paired donor splice site consensus sequence NGGTA(G)AG (SEQ ID NO: 01) in the codon NGG or the codon GGT or the codon GTA(G).

15. Use of the removal of non-paired donor splice sites in a part of a human or hamster codon-usage optimized nucleic acid sequence encoding an antibody, whereby the part is the part that encodes the heavy chain variable domain, for reducing mis-splicing or/and increasing antibody expression yield when said nucleic acid is used to produce the antibody in CHO cells.

16. The use according to claim 15, wherein non-paired splice sites are not removed in the part of the nucleic acid encoding the heavy chain constant region.

17. The use according to claim 15, wherein in the part of the nucleic acid encoding the light chain non-paired splice sites are removed.

18. The use according to claim 17, wherein the removal of the non-paired splice sites is by introducing an amino acid silent mutation in the nucleotide sequence NGGTA(G)AG (SEQ ID NO: 01).

19. The use according to claim 18, wherein the removal of the non-paired splice sites is by introducing an amino acid silent mutation in the nucleotide sequence NGGTA(G)AG (SEQ ID NO: 01) in the codon NGG or the codon GGT or the codon GTA(G).

20. The use according to claim 19, wherein the non-paired splice site is an artificial non-paired splice site.

21. The use according to claim 19, wherein the non-paired donor splice site is an artificial donor splice site and has been generated during codon optimization.


SEQUENCE LISTING (5 sequences)

SEQ ID NO: 1 - DNA, 6 bp, Artificial Sequence
  splice donor consensus sequence
ggtrag

SEQ ID NO: 2 - DNA, 8 bp, Artificial Sequence
  high GC content donor consensus sequence
aggtragt

SEQ ID NO: 3 - DNA, 8 bp, Artificial Sequence
  low GC content donor consensus sequence
aggtaagt

SEQ ID NO: 4 - DNA, 7 bp, Artificial Sequence
  splice acceptor sequence
  misc_feature (3)..(3): n is a, c, g, or t
ctnctag

SEQ ID NO: 5 - DNA, 39 bp, Artificial Sequence
  codon motif
gcggcggcggcggctgctgctgctgcagcagcagccgcc